(19) United States
(12) Patent Application Publication
(10) Pub. No.: US 2020/0197365 A1
(43) Pub. Date: Jun. 25, 2020

(54) TREATMENT WITH NMDA RECEPTOR MODULATORS

(71) Applicant: Naurex Inc., Madison, NJ (US)

(72) Inventors: Pradeep Banerjee, Hillsborough, NJ (US); John Donello, Dana Point, CA (US)

(21) Appl. No.: 16/210,056

(22) Filed: Dec. 5, 2018

Related U.S. Application Data
(60) Provisional application No. 62/594,891, filed on Dec. 5, 2017.

(51) Int. Cl. A61K 31/4025; A61K 9/00; A61P 25/20

(52) U.S. Cl. CPC A61K 31/4025; A61P 25/20; A61K 9/0019

(57) ABSTRACT

Disclosed are methods for treating a patient suffering from (i) a cognitive, neurological or psychological disease or disorder; and (ii) a sleep disorder comprising the step of administering to said patient a therapeutically effective amount of an N-Methyl-D-aspartic acid (NMDA) receptor partial agonist, in particular rapastinel or a pharmaceutically acceptable salt, ester, prodrug or metabolite thereof.

### BACKGROUND

An N-methyl-D-aspartate (NMDA) receptor is a postsynaptic, ionotropic receptor that is responsive to, inter alia, the excitatory amino acids glutamate and glycine and the synthetic compound NMDA. The NMDA receptor (NMDAR) appears to controls the flow of both divalent and monovalent ions into the postsynaptic neural cell through a receptor associated channel and has drawn particular interest since it appears to be involved in a broad spectrum of CNS disorders. The NMDAR has been implicated, for example, in neurodegenerative disorders including stroke-related brain cell death, convulsive disorders, and learning and memory. NMDAR also plays a central role in modulating normal synaptic transmission, synaptic plasticity, and excitotoxicity in the central nervous system. The NMDAR is further involved in Long-Term Potentiation (LTP), which is the persistent strengthening of neuronal connections that underlie learning and memory The NMDAR has been associated with other disorders ranging from hypoglycemia and cardiac arrest to epilepsy. In addition, there are preliminary reports indicating involvement of NMDA receptors in the chronic neurodegeneration of Huntington's, Parkinson's, and Alzheimer's diseases. Activation of the NMDA receptor has been shown to be responsible for post-stroke convulsions, and, in certain models of epilepsy, activation of the NMDA receptor has been shown to be necessary for the generation of seizures. In addition, certain properties of NMDA receptors suggest that they may be involved in the information-processing in the brain that underlies consciousness itself. Further, NMDA receptors have also been implicated in certain types of spatial learning.

In view of the association of NMDAR with various disorders and diseases, NMDA-modulating small molecule agonist and antagonist compounds have been developed for therapeutic use. NMDA receptor compounds may exert dual (agonist/antagonist) effect on the NMDA receptor through the allosteric sites. These compounds are typically termed “partial agonists”. In the presence of the principal site ligand, a partial agonist will displace some of the ligand and thus decrease Ca⁺⁺ flow through the receptor. In the absence of the principal site ligand or in the presence of a lowered level of the principal site ligand, the partial agonist acts to increase Ca⁺⁺ flow through the receptor channel.

Recently, a partial agonist of NMDAR with the following structure has been reported (rapastinel or GLYX-13):

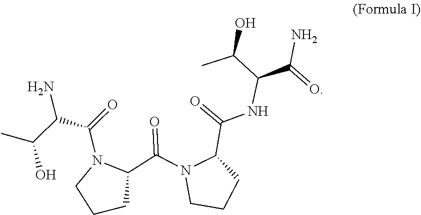

PCT/US2017/015851 describes a process for synthesis of peptide compounds, including rapastinel.

Major depressive disorder (MDD) is associated with abnormal sleep quality, including decreased slow-wave sleep (SWS) and dysregulated rapid eye movement (REM) sleep. Most currently approved antidepressants disrupt both SWS and REM sleep, likely due to increased levels of synaptic serotonin. (Nutt D, Wilson S, Paterson L. Sleep disorders as core symptoms of depression. Dialogues Clin Neurosci. 2008; 10(3):329-336). Increase in SWS is correlated with enhanced synaptic plasticity and is considered an electrophysiological correlate of improved mood. (Raven F, Van der Zee E A, Meerlo P, Havekes R. The role of sleep in regulating structural plasticity and synaptic strength: Implications for memory and cognitive function. Sleep Med Rev. 2017). Methods for treating depression and in particular MDD without significantly affecting sleep quality are advantageous.

### SUMMARY

Provided are methods for treating a patient suffering from (i) a cognitive, neurological or psychological disease or disorder; and (ii) a sleep disturbance or disorder comprising the step of administering to said patient a therapeutically effective amount of an N-Methyl-D-aspartic acid (NMDA) receptor partial agonist. In some embodiments, the NMDA receptor partial agonist is a compound of formula:

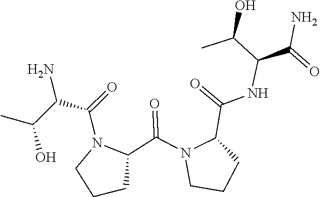

or a pharmaceutically acceptable salt, ester, metabolite or prodrug thereof.

In some embodiments, provided are methods for treating patients suffering from cognitive, neurological or psychological disease or disorder wherein said patients experience sleep disturbances or disorders. In some embodiments, the sleep disturbances and disorders are caused by the administration of one or more non-NMDA receptor partial agonists used for the treatment of cognitive, neurological or psychological diseases or disorders.

### BRIEF DESCRIPTION OF DRAWINGS

FIG. 1 Effect of Rapastinel, Ketamine, and Zolpidem on Latency to SWS and REM Sleep. FIG. 1 (A): Mean latency to SWS at baseline and following treatment. FIG. 1 (B): Mean latency to REM sleep at baseline and following treatment. *P<0.05, **P<0.01, ***P<0.001 versus baseline. REM, rapid eye movement; SWS, slow-wave sleep.

FIG. 2 Effects of Rapastinel, Ketamine, and Zolpidem on Duration (A) and

Depth (B) of SWS. FIG. 2 (A): Mean duration of SWS during the light period and dark period. FIG. 2 (B) Mean depth of SWS during the light period and dark period. *P<0.05, **P<0.01, ***P<0.001 versus baseline. SWS, slow-wave sleep.

FIG. 3 Effects of Rapastinel, Ketamine, and Zolpidem on Duration of REM Sleep. *P<0.05, ***P<0.001 versus baseline. REM, rapid eye movement.

FIG. 4 Effects of Rapastinel, Ketamine, and Zolpidem on Wakefulness. FIG. 4 (A): Mean duration of waking at baseline and following treatment. FIG. 4 (B): Mean percentage of active waking at baseline and following treatment. *P<0.05, **P<0.01, ***P<0.001 versus baseline.

### DETAILED DESCRIPTION

Provided herein are methods for treating a patient suffering from (i) a cognitive, neurological or psychological disease or disorder; and (ii) a sleep disturbance or disorder comprising the step of administering to said patient a therapeutically effective amount of an N-Methyl-D-aspartic acid (NMDA) receptor partial agonist. In some embodiments, the NMDA receptor partial agonist is a compound of formula:

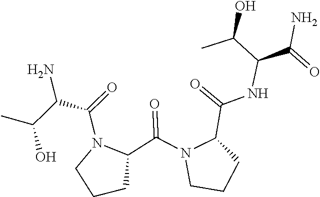

or a pharmaceutically acceptable salt, ester, metabolite or prodrug thereof.

In some embodiments, provided are methods for treating patients suffering from cognitive, neurological or psychological disease or disorder wherein said patients experience sleep disturbances or disorders. In some embodiments, the sleep disturbances and disorders are caused by the administration of one or more non-NMDA receptor partial agonists used for the treatment of cognitive, neurological or psychological diseases or disorders. Provided are methods for treatment of patients undergoing treatment for depression while also experiencing sleep disturbances and sleep disorders resulting from depression treatments. In some embodiments, patients are treated for major depressive disorder or refractory depression.

In some embodiments, patient is undergoing treatment with one more agents selected from a selective serotonin reuptake inhibitor (SSRI), serotonin-norepinephrine reuptake inhibitor (SNRI), serotonin modulator and stimulator (SMS), serotonin antagonist and reuptake inhibitor (SARI), norepinephrine reuptake inhibitor (NRI), norepinephrine-dopamine reuptake inhibitor (NDRI), tricyclic antidepressant (TCA), tetracyclic antidepressant (TeCA), monoamine oxidase inhibitor (MAOI) and atypical antipsychotic.

For example, SSRI can be selected from SSRI is selected from citalopram, escitalopram, paroxetine, fluoxetine, fluvoxamine and sertraline. In some embodiments, the patient has experienced sleep disturbances or sleep disorder as a result of treatment with SSRI. For example, SNRI can be selected from desvenlafaxine, duloxetine, levomilnacipran, milnacipran and venlafaxine. In some embodiments, the patient has experienced sleep disturbances or sleep disorder as a result of treatment with SNRI.

For example, SMS can be selected from vilazodone and vortioxetine. In some embodiments, the patient has experienced sleep disturbances or sleep disorder as a result of treatment with SMS. For example, atypical antipsychotics can be selected from amisulpride, aripiprazole, lurasidone, quetiapine, olanzapine, risperidone and ziprasidone. In some embodiments, the patient has experienced sleep disturbances or sleep disorder as a result of treatment with atypical antipsychotics.

In a preferred embodiment, provided are methods for treatment of major depressive disorder or refractive depression wherein said patient experiencing sleep disturbances or sleep disorder. In some embodiments, the patient is undergoing treatment with esketamine and has discontinued treatment with esketamine and experienced sleep disturbances or sleep disorders.

“Treating” includes any effect, e.g., lessening, reducing, modulating, or eliminating, that results in the improvement of the condition, disease, disorder and the like. “Individual,” “patient,” or “subject” are used interchangeably and include any animal, including mammals, preferably mice, rats, other rodents, rabbits, dogs, cats, swine, cattle, sheep, horses, or primates, and most preferably humans.

The term “effective amount” refers to an amount of the subject component, e.g., GLYX-13 (or a composition containing GLYX-13) that will elicit the biological or medical response of a tissue, system, animal or human that is being sought by the researcher, veterinarian, medical doctor or other clinician.

GLYX-13 may be obtained by recombinant or synthetic methods such as those described in U.S. Pat. Nos. 5,763,393 and 4,086,196 herein incorporated by reference. Also contemplated are polymorphs, hydrates, homologs, solvates, free bases, and/or suitable salt forms of GLYX 13 such as, but not limited to, the acetate salt. The peptide may be in cyclized or non-cyclized form as further described in U.S. Pat. No. 5,763,393. In some embodiments, a GLYX-13 analog may include an insertion or deletion of a moiety on one or more of the Thr or Pro groups such as a deletion of CH₂, OH, or NH₂ moiety. In other embodiments, GLYX-13 may be optionally substituted with one or more halogens, C₁-C₃ alkyl (optionally substituted with halogen or amino), hydroxyl, and/or amino. Other compounds contemplated for use herein include Glycine-site partial agonists of the NMDAR disclosed in U.S. Pat. Nos. 5,763,393, 6,107,271, and Wood et al., Neuro. Report, 19, 1059-1061, 2008, the entire contents of which are herein incorporated by reference.

In some embodiments, a therapeutically effective amount of GLYX-13 for adult human treatment administered, for example, during an induction period of time, are in the range of about 0.01 mg/kg to about 1000 mg/kg per administration (e.g., about 0.01 mg/kg to about 100 mg/kg, about 0.01 mg/kg to about 50 mg/kg, about 0.01 mg/kg to about 25 mg/kg, about 0.01 mg/kg to about 10 mg/kg, about 0.1 mg/kg to about 100 mg/kg, about 0.1 mg/kg to about 50 mg/kg, about 0.1 mg/kg to about 50 mg/kg, about 0.1 mg/kg to about 10 mg/kg, about 1 mg/kg to about 100 mg/kg, about 1 mg/kg to about 50 mg/kg, about 1 mg/kg to about 50 mg/kg per day, about 1 mg/kg to about 10 mg/kg, or about 1 mg/kg to about 10 mg/kg per administration, e.g., once a week, twice a week or three times a week and/or as described herein). The dosage of GLYX-13 may be at any dosage including, but not limited to, about 1 ug/kg, 25 ug/kg, 50 ug/kg, 75 ug/kg, 100 u ug/kg, 125 ug/kg, 150 ug/kg, 175 ug/kg, 200 ug/kg, 225 ug/kg, 250 ug/kg, 275 ug/kg, 300 ug/kg, 325 ug/kg, 350 ug/kg, 375 ug/kg, 400 ug/kg, 425 ug/kg, 450 ug/kg, 475 ug/kg, 500 ug/kg, 525 ug/kg, 550 ug/kg, 575 ug/kg, 600 ug/kg, 625 ug/kg, 650 ug/kg, 675 ug/kg, 700 ug/kg, 725 ug/kg, 750 ug/kg, 775 ug/kg, 800 ug/kg, 825 ug/kg, 850 ug/kg, 875 ug/kg, 900 ug/kg, 925 ug/kg, 950 ug/kg, 975 ug/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg. In certain embodiments, GLYX-13 may be therapeutically effective for depression with a range (e.g., an intravenous dose range) of about 1 to about10 mg/kg, e.g., about 5 to about10 mg/kg, e.g. about 1 mg/kg, about 5 mg/kg, or about 10 mg/kg.

In some embodiments, a therapeutically effective amount of GLYX-13 for adult human treatment administered, for example, during an induction period (administration period) of time may be a fixed dose of about 1000 mg to about 200 mg, or 900 mg to about 100 mg e.g., about 200 mg to about 500 mg, e.g., 50 mg, 100 mg, 225 mg, 250 mg, 200 mg, 300 mg, 350 mg, 450 mg, 500mg, 600mg, 700 mg, 750 mg, and/or 900 mg unit dose. It will be appreciated that a maintenance dose may be lower than the induction dose.

In some embodiments, any of the GLYX-13 dosages described herein can be administered on a less than daily basis, e.g., every other day (e.g., every two days); one or two times a week; one, two or three times a week; two or three times a week; twice weekly (e.g. every 3 days, every 4 days, every 5 days, every 6 days or e.g. administered with an interval of about 2 to about 3 days between doses); every three to four days; once a week; once every two weeks (bi-weekly); twice monthly; once a month or even less often. In certain embodiments, GLYX-13 is administered at a frequency of once a week, twice a week, once every two weeks, or any combination thereof.

In certain embodiments GLYX-13 (rapastinel) is administered at a range (e.g., an intravenous dose range) of about 1 to about 10 mg/kg, e.g., about 5 to about 10 mg/kg, e.g. about 1 mg/kg, about 5 mg/kg, or about 10 mg/kg, and/or GLYX-13 is administered at a frequency of once a week, once every two weeks, or any combination thereof.

In some embodiments, the methods and regimens include two or more treatment cycles (e.g. continuous cycles), in which each cycle includes an induction period of time and a rest period of time. As the skilled person will appreciate, each of the treatment cycles can be independently varied from one another in terms of dosage, frequency, duration of induction period of time, duration of rest period of time, etc.

### Administration and Formulations

GLYX-13 as well as any other pharmacological agent (e.g., one or more other antidepressant agents) of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations, or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In some embodiments, GLYX-13 herein may be administered parenterally to a patient including, but not limited to, subcutaneously, intramuscularly, and intravenously. In some embodiments, one or more of the components of the combinations described herein may also be administered via slow controlled i.v. infusion or by release from an implant device.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, intranasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, general safety and purity standards as required by FDA Office of Biologics standards. The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The combinations described herein may also contain other active compounds providing supplemental, additional, or enhanced therapeutic functions. Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

Disclosed compounds may be provided as part of a liquid or solid formulation, for example, aqueous or oily suspensions, solutions, emulsions, syrups, and/or elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl hydroxybenzoate and sorbic acid. Contemplated compounds may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water. For example, pharmaceutical formulations of rapastinel are disclosed in U.S. Patent Publication Nos. 20170296616 and 20170049844, incorporated by reference herein.

The present invention has multiple aspects, illustrated by the following non-limiting examples.

### EXAMPLES

### Rodent Surgery and Testing Procedure

Male rats were anesthetized and surgically implanted with 2 surface electrodes in the frontoparietal cortex and 2 depth electrodes in the CA1 region of the hippocampus (AP+5.0 mm, L±2.5 mm, V+7.0 mm). After ≥10 days recovery, telemetric transmitters were connected to electrodes for electroencephalographic (EEG) recordings. On the day of experiment, rats were injected at the beginning of the light phase with rapastinel (3, 10, or 30 mg/kg, IV), ketamine (30 mg/kg, IP), or the positive control zolpidem (12 mg/kg, PO) using a treatment schedule based on a cross-over design (n=9-10/dose/group) in which rats received zolpidem or rapastinel at session 1 and then rapastinel and/or ketamine at sessions 2-4. Physiological saline was used as the vehicle control

### Sleep-Wake Cycle Analysis

Sleep-wake cycles were measured continuously following rapastinel treatment for the next 23 hours and were analyzed based on a spectral power analysis. Latencies to first occurrence of SWS and REM sleep, as well as percent time spent in SWS, REM sleep, and wakefulness, were calculated for the total recording sessions (23 hours), the half cycles (0-12 and 12-23 hours), and 5 time segments (0-4, 4-8, 8-12, 12-18, and 18-23 hours). Depth of SWS (defined as the ratio between alpha and delta spectral power) was also calculated. Differences between vehicle and test drugs were calculated for all variables, for each animal, and for the means of the animals per treatment group, and analyzed using a paired t-test

### Results

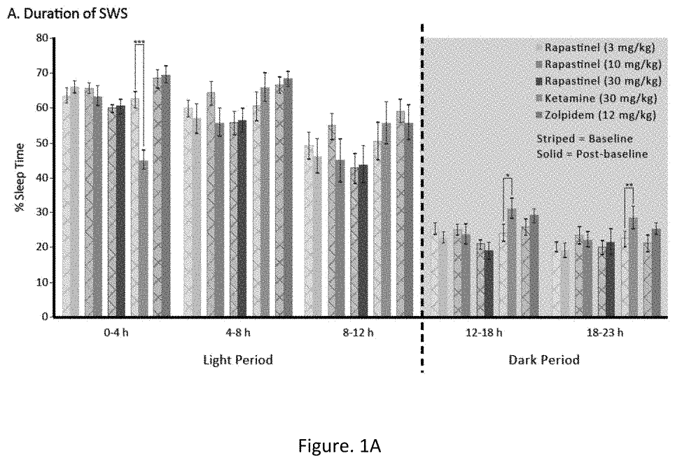

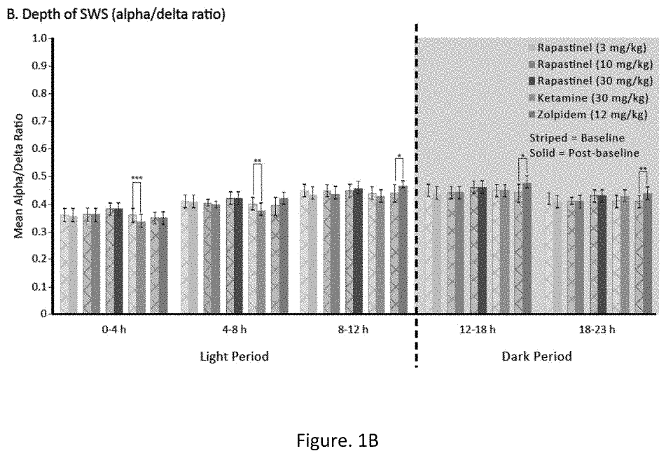

Rapastinel (3 mg/kg) slightly but significantly decreased latency to SWS onset (−33%; P<0.05) (FIG. 1A).

Higher doses of rapastinel (10 and 30 mg/kg) had no significant effect

Ketamine significantly increased (+393%; P<0.001) and zolpidem significantly decreased (−50%; P<0.05) latency to SWS onset (FIG. 1A).

Rapastinel (3, 10, and 30 mg/kg) had no significant effect on latency to REM sleep onset, whereas

ketamine and zolpidem significantly increased latency to REM sleep onset (ketamine: +104%, P<0.001; zolpidem: +67%; P<0.01) (FIG. 1B).

No drug had any significant effect on number of REM periods (P>0.05 for all).

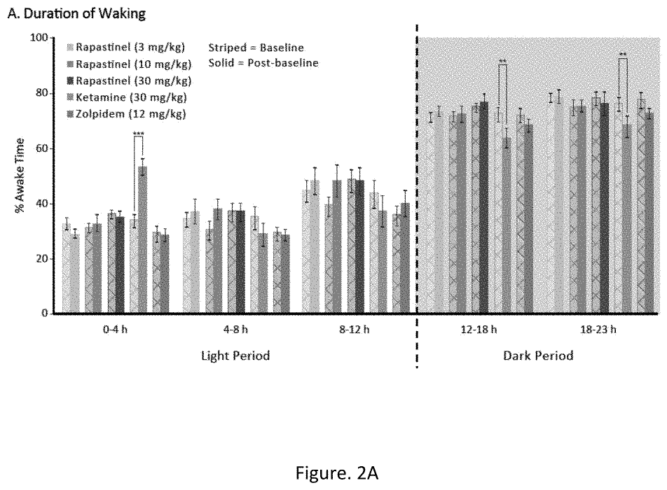

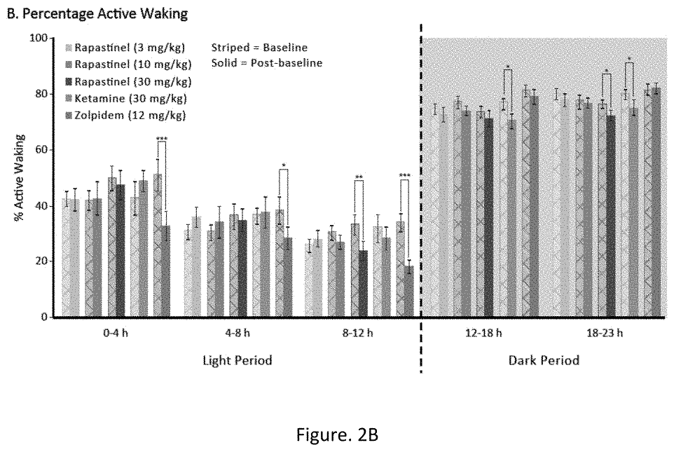

Rapastinel had no significant effect on duration or depth of SWS during the course of the entire recording session (FIG. 2).

Ketamine decreased duration of SWS (FIG. 2A) with a concomitant decrease in the alpha/delta ratio 0-4 hours post-dose (both, P0.001); 12-23 hours post-dose period, ketamine significantly increased SWS duration (P<0.05) (FIG. 2B).

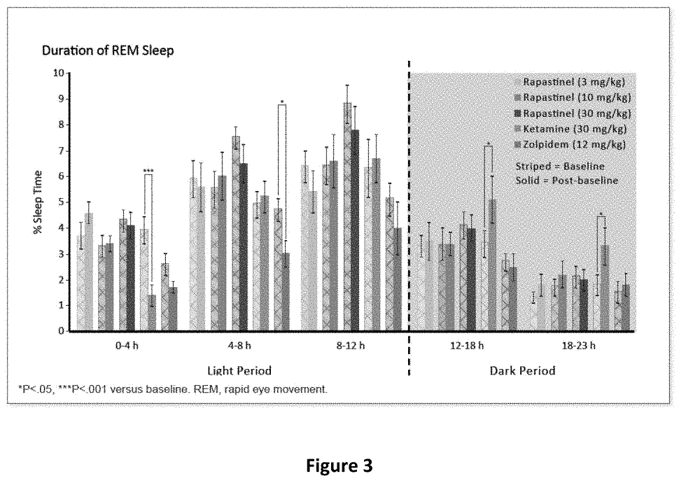

Zolpidem significantly increased depth of SWS>8 hours post-dose (P<0.05) (FIG. 2B). Rapastinel (3, 10, and 30 mg/kg) did not have a significant effect on the duration of REM sleep (FIG. 3).

Ketamine decreased the duration of REM sleep 0-4 hours post-dose (P<0.001), which was followed by a compensatory increase in REM sleep 12-23 hours post-dose (P<0.05) (FIG. 3).

Zolpidem significantly decreased the duration of REM sleep 4-8 hours post-dose (P<0.05) (FIG. 3).

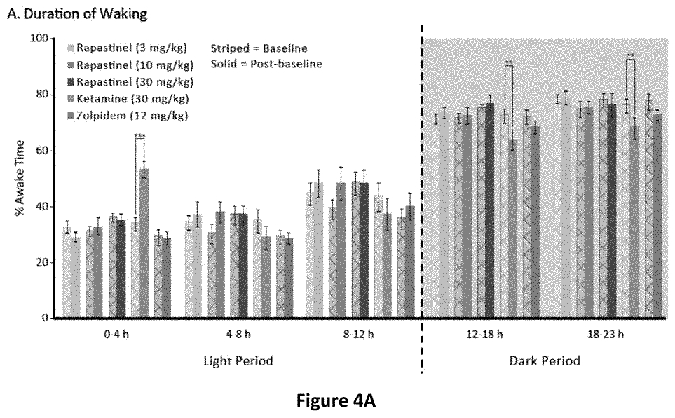

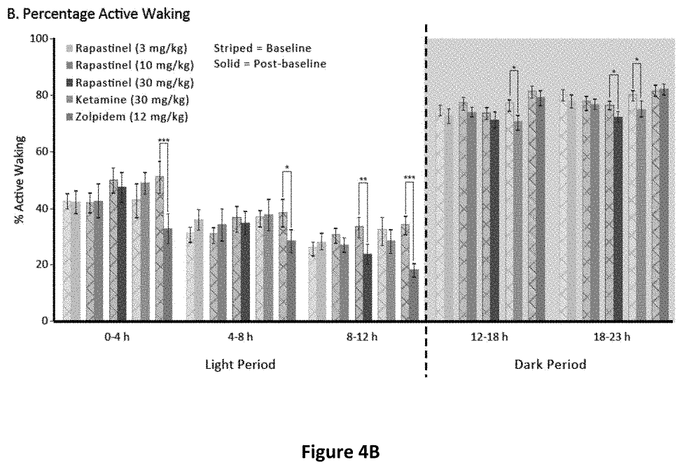

Rapastinel (3, 10, or 30 mg/kg) and zolpidem had no effect on the duration of waking, whereas ketamine significantly increased the duration of waking during the first 4 hours post-dose (P<0.001); 12-23 hours post-dose, ketamine-treated rats exhibited a small but significant decrease in waking (P<0.01) (FIG. 4A).

Rapastinel had no effect on the percentage of active waking, with the exception of a small but significant decrease between 8-12 hours (P<0.01) and between 18-23 hours (P<0.05) post-dose at 30 mg/kg (FIG. 4B).

Ketamine and zolpidem significantly decreased percentage of active waking 12-23 hours (P<0.05) and 0-12 hours (P<0.05) post-dose, respectively (FIG. 4B).

1. A method of treating a patient suffering from (i) a cognitive, neurological or psychological disease or disorder; and (ii) a sleep disorder or sleep disturbances, comprising the step of administering to said patient a therapeutically effective amount of an N-Methyl-D-aspartic acid (NMDA) receptor partial agonist.

2. The method according to claim 1 wherein said sleep disorder is selected from an insomnia, a primary insomnia, an idiopathic insomnia; an insomnia associated with depression, emotional/mood disorders, aging, Alzheimer's disease or cognitive impairment; REM sleep interruptions; a breathing-related sleep disorder; a sleep apnea; a periodic limb movement disorder, a restless leg syndrome; a circadian rhythm sleep disorder; a shift work sleep disorder; and a jet-lag syndrome.

3. The method according to claim 1 wherein said cognitive, neurological or psychological disease or disorder is selected from the group consisting of deficiency in memory, intellect, or learning and logic ability; reduction in any particular individual's functioning in one or more cognitive aspects; age-related cognitive decline; dementia; Alzheimer's disease; multi-infarct dementia; alcoholic dementia or other drug-related dementia; dementia associated with intracranial tumors or cerebral trauma; dementia associated with Huntington's disease or Parkinson's disease; AIDS-related dementia; delirium; amnestic disorder; mental retardation; a learning disorder including reading disorder, mathematics disorder, or a disorder of written expression; attention-deficit/hyperactivity disorder; schizophrenia, schizophrenia including negative symptoms; schizophreniform disorder; schizoaffective disorder, schizoaffective disorder of the delusional type, schizoaffective disorder of the depressive type; delusional disorder; substance-induced psychotic disorder; personality disorder of the paranoid type; personality disorder of the schizoid type; panic disorder; phobias; obsessive-compulsive disorder; stress disorders; generalized anxiety disorder; movement disorders involving Huntington's disease; dyskinesia associated with dopamine agonist therapy; Parkinson's disease: restless leg syndrome; disorders comprising as a symptom thereof a deficiency in cognition.

4. The method according to claim 1, wherein said patient is undergoing treatment with one or more agents that enhance the release, production and/or functioning of serotonin, dopamine, epinephrine, norepinephrine, and glutamate neurotransmitters for the treatment of said a cognitive, neurological or psychological disease or disorder.

5. The method according to claim 1, wherein said patient is undergoing treatment with one or more agents selected from selective serotonin reuptake inhibitors (SSRI), serotonin agonists, antagonists and modulators, selective norepinephrine reuptake inhibitors (SNRIs).

6. The method according to claim 1 wherein said patient is undergoing treatment with one or more agents selected from opiate agonists, opiate antagonists, opiate partial agonists, calcium channel antagonists, SHT, 5-HT_{1A} complete or partial receptor agonists or antagonists, 5-HT_{2A} complete or partial receptor agonists or antagonists, 5-HT₃ complete or partial receptor agonists or antagonists, sodium channel antagonists, N-methyl-D-aspartate (NMDA) receptor antagonists, COX-2 selective inhibitors, neurokinin receptor 1 (NK1) antagonists, non-steroidal anti-inflammatory drugs (NSAID), selective serotonin reuptake inhibitors (SSRI) and/or selective serotonin and norepinephrine reuptake inhibitors (SSNRI), tricyclic antidepressant drugs, norepinephrine modulators, lithium, valproate, norepinephrine reuptake inhibitors, monoamine oxidase inhibitors (MAOIs), reversible inhibitors of monoamine oxidase (RIMAs), alpha-adrenoreceptor antagonists, atypical anti-depressants, benzodiazepines, corticotropin releasing factor (CRF) antagonists, neurontin (gabapentin) and pregabalin.

7. The method according to claim 1 wherein said patient is undergoing treatment with lithium or valproate.

8. The method according to claim 1 wherein said patient is undergoing treatment with ketamine or esketamine.

9. The method according to claim 1 wherein said patient is undergoing treatment with one more agents selected from a selective serotonin reuptake inhibitor (S SRI), serotonin-norepinephrine reuptake inhibitor (SNRI), serotonin modulator and stimulator (SMS), serotonin antagonist and reuptake inhibitor (SARI), norepinephrine reuptake inhibitor (NRI), norepinephrine-dopamine reuptake inhibitor (NDRI), tricyclic antidepressant (TCA), tetracyclic antidepressant (TeCA), monoamine oxidase inhibitor (MAOI) and atypical antipsychotic.

10. The method according to claim 9 wherein said SSRI is selected from citalopram, escitalopram, paroxetine, fluoxetine, fluvoxamine and sertraline.

11. The method according to claim 9 wherein said SNRI is selected from desvenlafaxine, duloxetine, levomilnacipran, milnacipran and venlafaxine.

12. The method according to claim 9 wherein said SMS is selected from vilazodone and vortioxetine.

13. The method according to claim 9 wherein said SARI is selected from nefazodone and trazodone.

14. The method according to claim 9 wherein said NRI is selected from reboxetine, Teniloxazine and viloxazine.

15. The method according to claim 9 wherein said NDRI is selected from bupropion, amineptine, lisdexamfetamine, and methylphenidate.

16. The method according to claim 9 wherein said TCA is selected from amitriptyline amitriptylinoxide, clomipramine, desipramine, dibenzepin, dimetacrine, dosulepin doxepin, imipramine, lofepramine, melitracen, nitroxazepine, nortriptyline, noxiptiline, opipramol, pipofezine, protriptyline and trimipramine.

17. The method according to claim 9 wherein said TeCA is selected from amoxapine, maprotiline, mianserin, mirtazapine, setiptiline.

18. The method according to claim 9 wherein said atypical antipsychotics is selected from amisulpride, aripiprazole, lurasidone, quetiapine, olanzapine, risperidone and ziprasidone.

19. (canceled)

20. (canceled)

21. The method according to claim 1 wherein said cognitive disease or disorder is selected from: deficiency in memory, intellect, learning, logic ability; reduction in any particular individual's functioning in one or more cognitive aspects; age-related cognitive decline;
dementia; Alzheimer's disease; multi-infarct dementia; alcoholic dementia or other drug-related dementia; dementia associated with intracranial tumors or cerebral trauma; dementia associated with Huntington's disease or Parkinson's disease; AIDS-related dementia; delirium; amnestic disorder; mental retardation; a learning disorder including reading disorder, mathematics disorder, or a disorder of written expression;
attention-deficit/hyperactivity disorder; schizophrenia including negative symptoms; schizophreniform disorder; schizoaffective disorder including of the delusional type or the depressive type; delusional disorder; substance-induced psychotic disorder; personality disorder of the paranoid type; personality disorder of the schizoid type; panic disorder; phobias; obsessive-compulsive disorder; stress disorders; generalized anxiety disorder; movement disorders involving Huntington's disease; dyskinesia associated with dopamine agonist therapy; Parkinson's disease: restless leg syndrome; disorders comprising as a symptom thereof a deficiency in cognition.

22. The method according to claim 1 wherein said NMDA receptor partial agonist is a compound of formula: 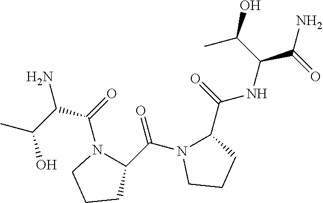
or a pharmaceutically acceptable salt, ester, metabolite or prodrug thereof.

23. The method according to claim 22 wherein said compound is administered at a dose of about 5 mg/kg or about 10 mg/kg or about 15 mg/kg weekly or every other week for six to twelve weeks.

24. The method according to claim 22 wherein said compound is administered at dosage of about 200 mg to about 1000 mg per week or every other week for at least four weeks in one cycle followed by at least one week, two weeks, three weeks, four weeks, two months or more where said compound is not administered.

25. The method according to claim 22 wherein said compound is administered at dosage of about 225 mg per week or every other week for at least four weeks in one cycle followed by at least one week, two weeks, three weeks, four weeks, two months or more where said compound is not administered.

26. The method according to claim 22 wherein said compound is administered at dosage of about 450 mg per week or every other week for at least four weeks in one cycle followed by at least one week, two weeks, three weeks, four weeks, two months or more where said compound is not administered.

27. The method according to claim 22 wherein said compound is administered at dosage of about 900 mg per week or every other week for at least four weeks in one cycle followed by at least one week, two weeks, three weeks, four weeks, two months or more where said compound is not administered.

28. The method according to claim 22 wherein said compound is administered intravenously.

29. The method according to claim 1 wherein said sleep disorder is caused by the administration of one or more agents selected from opiate agonists, opiate antagonists, opiate partial agonists, calcium channel antagonists, 5HT, 5-HT_{1A} complete or partial receptor agonists or antagonists, 5-HT_{2A} complete or partial receptor agonists or antagonists, 5-HT₃ complete or partial receptor agonists or antagonists, sodium channel antagonists, N-methyl-D-aspartate (NMDA) receptor antagonists, COX-2 selective inhibitors, neurokinin receptor 1 (NK1) antagonists, non-steroidal anti-inflammatory drugs (NSAID), selective serotonin reuptake inhibitors (SSRI) and/or selective serotonin and norepinephrine reuptake inhibitors (SSNRI), tricyclic antidepressant drugs, norepinephrine modulators, lithium, valproate, norepinephrine reuptake inhibitors, monoamine oxidase inhibitors (MAOIs), reversible inhibitors of monoamine oxidase (RIMAs), alpha-adrenoreceptor antagonists, atypical anti-depressants, benzodiazepines, corticotropin releasing factor (CRF) antagonists, neurontin (gabapentin) and pregabalin.

30. The method according to claim 1 wherein said sleep disorder is caused by the administration of lithium or valproate.

31. The method according to claim 1 wherein said sleep disorder is caused by the administration of ketamine or esketamine.

32. The method according to claim 1 wherein said sleep disorder is caused by the administration of a selective serotonin reuptake inhibitor (SSRI), serotonin-norepinephrine reuptake inhibitor (SNRI), serotonin modulator and stimulator (SMS), serotonin antagonist and reuptake inhibitor (SARI), norepinephrine reuptake inhibitor (NRI), norepinephrine-dopamine reuptake inhibitor (NDRI), tricyclic antidepressant (TCA), tetracyclic antidepressant (TeCA), monoamine oxidase inhibitor (MAOI) and atypical antipsychotic.

33. The method according to claim 32 wherein said SSRI is selected from citalopram, escitalopram, paroxetine, fluoxetine, fluvoxamine and sertraline.

34. The method according to claim 32 wherein said SNRI is selected from desvenlafaxine, duloxetine, levomilnacipran, milnacipran and venlafaxine.

35. The method according to claim 32 wherein said SMS is selected from vilazodone and vortioxetine.

36. The method according to claim 32 wherein said SARI is selected from nefazodone and trazodone.

37. The method according to claim 32 wherein said NRI is selected from reboxetine, Teniloxazine and viloxazine.

38. The method according to claim 32 wherein said NDRI is selected from bupropion, amineptine, lisdexamfetamine, and methylphenidate.

39. The method according to claim 32 wherein said TCA is selected from amitriptyline amitriptylinoxide, clomipramine, desipramine, dibenzepin, dimetacrine, dosulepin doxepin, imipramine, lofepramine, melitracen, nitroxazepine, nortriptyline, noxiptiline, opipramol, pipofezine, protriptyline and trimipramine.

40. The method according to claim 32 wherein said TeCA is selected from amoxapine, maprotiline, mianserin, mirtazapine, setiptiline.

41. The method according to claim 32 wherein said atypical antipsychotics is selected from amisulpride, aripiprazole, lurasidone, quetiapine, olanzapine, risperidone and ziprasidone.

42. The method according to claim 1 wherein said sleep disorder is selected from insomnia, REM sleep interruptions, breathing-related sleep disorders, sleep apnea, periodic limb movement disorders, restless leg syndromes, circadian rhythm sleep disorders and shift work sleep disorders.

43. The method according to claim 1 wherein said sleep disturbances or disorders are correlated to an increase in synaptic serotonin wherein said increase is caused by the administration of an agent used for the treatment of a neurological, psychological or cognitive disorder.

44. The method according to claim 30 wherein said neurological, psychological or cognitive disease or disorder is depression, MDD or refractory depression.
